(19) United States
(12) Patent Application Publication
(10) Pub. No.: US 2015/0174245 A1
(43) Pub. Date: Jun. 25, 2015

(54) TRANSPARENT GEL

(71) Applicant: Thomas RIESINGER, Nurnberg (DE)

(72) Inventor: Thomas RIESINGER, Nurnberg (DE)

(73) Assignee: Nawa Heilmittel GmbH, Nurnberg (DE)

(21) Appl. No.: 14/578,604

(22) Filed: Dec. 22, 2014

(30) Foreign Application Priority Data
Dec. 23, 2013 (DE) 20 2013 105 935.8
Jan. 24, 2014 (DE) 20 2014 100 305.3

(51) Int. Cl. A61K 47/02; A61K 31/16; A61K 47/36; A61K 31/155

(52) U.S. Cl. CPC A61K 47/02; A61K 31/155; A61K 31/16; A61K 47/36

(57) ABSTRACT

The present invention describes a transparent gel containing at least one gelling agent and an aqueous sulfuric acid solution containing 10 mg to 50 mg zinc and 5 mg to 35 mg iron, each based on 1 kg gel. The aqueous solution has a pH between 3.0 and 2.0 and contains 100 mg to 1000 mg polyhexamethylene biguanide (PHMB) based on 1 kg gel.

### TECHNICAL FIELD

The invention relates to a transparent gel.

### STATE OF THE ART

Zinc oxide is used to prevent and treat inflammatory changes of the skin and the subcutaneous tissue. GB 20 22 998 A for example discloses a pharmaceutical preparation consisting of an aqueous solution of at least one metallic trace element or a salt of such a trace element, wherein the pH of the solution is set at a value of less than 4. EP 363 696 A1 prescribes a pharmaceutical preparation consisting of an aqueous solution of metallic trace elements in the form of zinc and iron as well as sulfuric acid as the physiological salt. This solution has a pH between 3.0 and 2.0 and contains zinc, iron and sulfuric acid in certain proportions.

The term “herpes simplex” refers to a viral infection caused by herpes simplex viruses. The word “herpes” is used conversationally for such an infection. The pathogens of herpes simplex infections are two different viral species: herpes simplex virus type 1 (HSV 1) and herpes simplex virus type 2 (HSV 2). These show minor deviations with regard to their signs and symptoms and localization of the disease. Clinically different HSV infections are identified according to the localization of the symptoms, of which there is a high incidence of both herpes simplex labialis (herpes of the lips, i.e., oral herpes) and herpes simplex genitalis (genital herpes).

Oral herpes is treated primarily with acyclovir and valacyclovir. Acyclovir is also offered as a cream in addition to tablet form. The known pharmaceutical preparations often lead to a standstill in the development of the disease symptoms when oral herpes is treated but they cannot induce accelerated healing.

### SUMMARY OF THE INVENTION

The purpose of the invention is to provide a composition achieving an accelerated healing of oral herpes. This problem is solved according to the invention by the transparent gel according to claim 1. Additional advantageous aspects, details and embodiments of the invention are derived from the dependent claims and the description.

A gel in the sense of the present invention is a finely dispersed system comprised of a solid phase and a liquid phase. The solid phase forms a spongy three-dimensional network whose pores are filled by a liquid.

The present invention makes available a transparent gel containing at least one gelling agent and an aqueous sulfuric acid solution containing 10 mg to 50 mg zinc and 5 mg to 35 mg iron, each based on 1 kg gel. The aqueous solution has a pH between 3.0 and 2.0 and contains 100 mg to 1000 mg polyhexamethylene biguanide (PHMB) based on 1 kg gel.

It has surprisingly been found that the gel according to the invention not only stops the development of the disease symptoms but also causes accelerated healing of the symptoms of oral herpes. Because of its transparency properties, the gel is invisible on the herpes blisters, in contrast with creams, and is not attracting attention.

The antiseptic polyhexamethylene biguanide (PHMB), also known as polyhexanide or polyaminopropyl biguanide, is a polymer biguanide with antimicrobial properties.

The gel preferably also contains dexpanthenol. Dexpanthenol has antipruritic, anti-inflammatory, wound-healing and skin care properties and, when combined with the gel according to the invention, has proven to be particularly suitable for accelerating the healing of disease symptoms of oral herpes.

According to another preferred embodiment, the gel additionally contains at least one preservative, wherein one or more preservatives are preferably selected from the group consisting of benzyl alcohol, methylchloroisothiazoleinone and methylisothiazolinone as the preservative.

The gel preferably contains zinc in the form of zinc chloride and iron in the form of iron chloride.

The gel preferably contains 150 mg to 500 mg polyhexamethylene biguanide (PHMB) based on 1 kg gel. The gel particularly preferably contains 200 mg to 300 mg polyhexamethylene biguanide (PHMB) based on 1 kg gel.

Various substances such as hydroxypropyl starch phosphate or dehydroxanthan gum have been investigated as gelling agents. Experimental tests have shown that dehydroxanthan gum is particularly suitable as a gelling agent. It forms a dimensionally-stable, transparent gel, which produces a pleasant feeling on the skin, adheres adequately and leaves no residues on the skin after drying. Dehydroxanthan gum has viscosity and thermal stability while also having the advantage that the gel remains stable and does not flow during application.

The gel preferably contains 100 mg to 160 mg sulfuric acid based on 1 kg gel. This amount of sulfuric acid is suitable for adjusting the desired pH between 3.0 and 2.0.

The gel particularly preferably contains 20 mg to 40 mg zinc and 10 mg to 30 mg iron, each based on 1 kg gel. In particular the gel preferably contains 30 mg zinc and 19 mg iron, each based on 1 kg gel. The preferred embodiments mentioned above result in a further accelerated healing of the disease symptoms of oral herpes.

The gel according to the invention is tolerated extremely well and is characterized by good mucous membrane tolerability and ensures a high bioavailability of the metallic trace elements zinc and iron, so that the desired therapeutic effect is achieved at even low doses.

It has been found that the combination of metallic trace elements in the form of zinc and iron as well as aqueous sulfuric acid and an amount of PHMB lead to very surprising unexpected effects, namely rapid and complete healing of symptoms of oral herpes.

With the gel according to the invention, water may be used in the form of distilled water or electrolytically demineralized water.

One embodiment of the preparation according to the invention, with which very good results are achieved, has concentrations of 30 mg zinc in the form of zinc chloride and 19 mg iron in the form iron chloride per kg gel.

Another factor for the efficacy of the gel according to the invention is the pH, which is in the range between 3.0 and 2.0 for the desired effect. The pH is adjusted by adding a corresponding amount of sulfuric acid.

The gel according to the present invention may also contain, in addition to the metallic trace elements iron and zinc, nonmetallic trace elements, for example, from the group of silicon, iodine and fluorine, which are present in the preparation in a physiological amount or a multiple thereof.

Essentially a wide variety of methods are suitable for production of the preparation according to the invention. Thus, for example, the preparation may take place in such a way that the metallic trace elements iron and zinc are preferably dissolved in pulverized or powdered form with acid and water, whereupon the pH of the solution is adjusted by the added amount of water and by the added amount of sulfuric acid.

Furthermore, it is also possible to produce the preparation according to the invention by dissolving water-soluble metal compounds of the metallic trace elements, namely zinc chloride and iron chloride in water with the addition of sulfuric acid, but again in this case the required pH is adjusted through the added amount of water and sulfuric acid.

A gel according to the present invention is preferably produced by a method comprising the steps of preparing an aqueous sulfuric acid solution containing 10 mg to 50 mg zinc, 5 mg to 35 mg iron and 100 mg to 1000 mg polyhexamethylene biguanide (PHMB), each based on 1 kg gel, wherein the aqueous solution has a pH between 3.0 and 2.0, and addition of a gelling agent to the aqueous sulfuric acid solution while stirring. Preferably, the step of addition of a preservative to the resulting solution is additionally carried out.

### MEANS OF IMPLEMENTING THE INVENTION

To prepare a gel according to the present invention, first 30 mg zinc in the form zinc chloride and 19 mg iron in the form of iron chloride are dissolved in demineralized water. Then 150 mg polyhexamethylene biguanide (PHMB) and dexpanthenol are added. After each addition, the solution is adjusted to the desired pH of 2.5 with the help of dilute sulfuric acid. Next the dehydroxanthan gum gelling agent is introduced into the aqueous solution while stirring slowly. After the gel has developed in the desired form, methylchloroisothiazolinone is added as a preservative to improve the stability of the gel.

1. A transparent gel containing at least one gelling agent and an aqueous sulfuric acid solution containing 10 mg to 50 mg zinc and 5 mg to 35 mg iron, each based on 1 kg gel, wherein the aqueous solution has a pH between 3.0 and 2.0, characterized in that it contains 100 mg to 1000 mg polyhexamethylene biguanide (PHMB), based on 1 kg gel.

2. The gel according to claim 1, characterized in that it also contains dexpanthenol.

3. The gel according to claim 1, characterized in that it additionally contains at least one preservative, wherein the preservative is preferably one or more preservatives selected from the group consisting of benzyl alcohol, methylchloroisothiazolinone and methylisothiazolinone.

4. The gel according to claim 1, characterized in that it additionally contains zinc in the form of zinc chloride and iron in the form of iron chloride.

5. The gel according to claim 1, characterized in that it also contains 150 mg to 500 mg polyhexamethylene biguanide (PHMB), based on 1 kg gel.

6. The gel according to claim 1, characterized in that it also contains 200 mg to 300 mg polyhexamethylene biguanide (PHMB), based on 1 kg gel.

7. The gel according to claim 1, characterized in that it contains dehydroxanthan gum as the gelling agent.

8. The gel according to claim 1, characterized in that it contains 100 mg to 160 mg sulfuric acid, based on 1 kg gel.

9. The gel according to claim 1, characterized in that the aqueous solution contains 20 mg to 40 mg zinc and 10 mg to 30 mg iron, each based on 1 kg gel.

10. The gel according to claim 9, characterized in that it contains 30 mg zinc and 19 mg iron, each based on 1 kg gel.
